(19) United States
(12) Patent Application Publication
(10) Pub. No.: US 2008/0208300 A1
(43) Pub. Date: Aug. 28, 2008

(54) Ionically conductive neural bridge

(75) Inventor: Nicholas F. Pasch, S. E. Bellevue, CA (US)

Correspondence Address: NICHOLAS F. PASCH, 2706 162ND AVENUE, S.E. BELLEVUE, WA, 98008, US

(73) Assignee: Seertech Corporation, Charlotte, NC (US)

(21) Appl. No.: 11/474,107

(22) Filed: Jun. 23, 2006

(51) Int. Cl. A61N 1/02

(52) U.S. Cl. 607/116

(57) ABSTRACT

Neural bridge devices for providing ionic communication across damaged or separated portions of a neuron, or between a neuron and an electronic device, are disclosed. The neural bridge devices can include an ionically conductive polymer that may functionally replace the biological conduction of action potentials along an axon, to restore sensory or motor nerve function, and may enhance neuronal healing.

### FIELD OF THE INVENTION

This invention generally relates to devices and methods for repairing damaged nerves and for establishing interfaces among nerves, other biological cell types and electronic devices.

### BACKGROUND OF THE INVENTION

Nerve damage is a common result of crushing and cutting injuries, as well as occurring as a result of some diseases, chemical exposures and degenerative disorders. Whether impacting the central nervous system or individual peripheral nerves, nerve damage may be associated with losses of sensory or motor function, is often slow to heal, and in many cases is considered to be irreversible. In addition, damaged nerves may not heal in a manner that restores sensory or motor function. For example, if a nerve cell is severed, the separated portions may not heal toward one another to rejoin, especially if separated by a substantial distance.

Individual nerve cells (neurons) include a cell body (soma), dendrites that receive stimuli from an outside source such as another neuron, and an axon along which nerve impulses propagate from the soma to terminal branches for conveying the nerve impulse to another cell, for example, to another neuron or to a muscle cell. In a resting state, a neuron maintains ionic concentration gradients (an action potential) across its cell membrane along the axon. For example, the resting concentration of potassium ions is much higher inside the axon (in the axoplasm) than outside (in the extracellular fluid), while the concentration of sodium ions is much higher in the extracellular fluid the axon than in the axoplasm. Some axons are sheathed with myelin, formed of a type of cell called Schwann cells. For these axons, the action potential is established at periodic discontinuities along the sheath, called Nodes of Ranvier, while the myelin acts as an insulator about the axon between the nodes.

The nerve impulses propagate along the axon in a wavelike fashion, associated with transient changes in the action potential, which is restored to its resting state by the cell after an impulse passes. The time during which the action potential is reestablished is referred to as a refractory period, during which additional nerve impulses are not transmitted. The refractive period is also responsible for the transmission of nerve impulses in only one direction along the axon.

An axon may exceed a meter in length, may extend between widely separated parts of the body, and axonal damage is frequently suffered with physical trauma to portions of the body through which the axon passes. Many efforts have been made to develop methods and devices for repairing damaged axons. These efforts have included, individually and in combination, tissue grafts to replace damaged or cut segments of axons, chemical and biochemical agents to stimulate healing, mechanical devices to rejoin and support severed axons, bridging devices such as tubes for guiding axonal healing, and the application of energy to directly rejoin damaged tissue or to stimulate healing in desired directions. In addition, interfaces between nerves and electronic devices are being investigated as means to reestablish functionality across damaged nerves and to develop interfaces between nerves and external devices.

Although moderate successes have been achieved in enhancing the healing of nerves, nerve damage remains one of the most difficult types of injury to treat successfully, and many nerve injuries remain untreatable.

### SUMMARY OF THE INVENTION

Embodiments of the present invention pertain to neural repair devices and methods for their application. One aspect of the present invention includes an ion conduit. The ion conduit can include an elongated central member with a first end, a second end, a central axis and a substantially cylindrical outer surface about the axis. In other embodiments, the central member can include a non-cylindrical outer surface, or may be branched. The central member can be adapted to conduct ions along the axis between the first end and the second end. An ionically insulating jacket can surround at least a portion of the outer surface. At least one of the first and second ends can be adapted to be ionically coupled to a neuron of a living animal, such as to effect the transport of ions between the central member and the neuron. In an embodiment, at least one of the first and second ends of the ion conduit can be coupled to a severed end of a nerve.

In one embodiment, the central member can include an ionically conductive polymer. A copolymer such as a block copolymer can be used, for example. In another embodiment, the conductive polymer can be methoxy polyethylene glycol methacrylate. Ions conducted by the conduit can include sodium ions, potassium ions, and/or calcium ions. Either or both of the central member and the jacket can be constructed of a biocompatible polymer or a bioabsorbable polymer, for example. The ion conduit can also include a recess in at least one of the first and second ends for receiving a portion of the neuron.

The ion conduit can further include one or more interruptions in the jacket for coupling ions or electrical current through the outer surface of the central member. Also, one or more electrode can be electrically coupled to at least one of the central member and the jacket. In various embodiments, the one or more electrode may be coupled to an electrical interface that can convert between an ionic current and an electrical current. The ion conduit can also include a sensor for sensing an action potential within the central member. In addition, one or more of the central member and the insulating jacket can be adapted to transport a cellular nutrient between a nutrient source and the neuron, or between severed ends of a neuron.

Another aspect of embodiments of the present invention involves an implant for bridging a gap in a severed nerve of a living being. The implant can include at least one ionically conductive strand having a first end, a second end, an ionically conductive polymeric core, and an ionically insulating jacket about at least a portion of the core. At least one of the first and second ends can be adapted to be ionically coupled to the severed nerve. The implant can include any number of strands. In one embodiment, the implant can includes at least two ionically conductive strands. In various embodiments, the individual ionically conductive strands may be adapted to conduct ions in a direction substantially away from the central nervous system, toward the central nervous system, or adjustably in either direction. In one embodiment, the implant can include one or more electrode for modifying an action potential in one or more strand of the implant.

Yet another aspect of embodiments of the present invention involves a neural bandage. The neural bandage can include a multilayer sheet having an ionically conductive layer and an ionically insulating layer. The sheet may be flexible enough to be wrapped about a damaged portion of a neuron, and can includes an adhesive for maintaining the position of the applied bandage when portions of the bandage are overlapped about the damaged neuron, for example.

Still another aspect of embodiments of the present invention involves a method for repairing damage to a neuron of a living animal. The method can provide an elongated ion conduit having a first end, a second end, and a length therebetween, an ionically conducting portion and an ionically insulating portion, each extending along the length substantially from the first end to the second end. In one embodiment, the method can also include positioning the ion conduit to ionically connect undamaged portions of the neuron, thereby bridging the damage. Positioning the conduit can include connecting at least one end of the ion conduit to a severed end of the neuron. Positioning the conduit can also include establishing a passage through which the neuron can grow to reconnect a first severed of the neuron to a second severed end of the neuron.

### BRIEF DESCRIPTION OF THE DRAWINGS

Aspects and features of the present invention may be better understood by referring to the following description in conjunction with the accompanying drawings and claims, in which like numerals indicate like structural elements and features in various figures. The drawings are not necessarily to scale, emphasis instead being placed upon illustrating embodiments and features of the invention.

FIG. 1 illustrates an embodiment of an ion conduit in accordance with embodiments of the present invention.

FIG. 2 illustrates the ion conduit of FIG. 1, bridging a gap in a damaged axon.

FIG. 3 illustrates an embodiment of an ion conduit including a cannulation in accordance with embodiments of the present invention.

FIGS. 4a and 4b illustrate an ion conduit with a multilayer neural bandage in accordance with embodiments of the present invention.

FIG. 5 illustrates ion conduits including one or more interface to external devices in accordance with embodiments of the present invention.

### DESCRIPTION OF THE PREFERRED EMBODIMENTS

Certain exemplary embodiments will now be described to provide an overall understanding of the aspects and features of the devices and methods of use disclosed herein. Examples of these embodiments and features are illustrated in the drawings. Those of ordinary skill in the art will understand that the devices and methods of use disclosed herein can be adapted and modified to provide other devices and methods for other applications and that other additions and modifications can be made without departing from the scope of the present disclosure. For example, the features illustrated or described as part of one embodiment or one drawing can be used on another embodiment or another drawing to yield yet another embodiment. Such modifications and variations are intended to be included within the scope of the present disclosure.

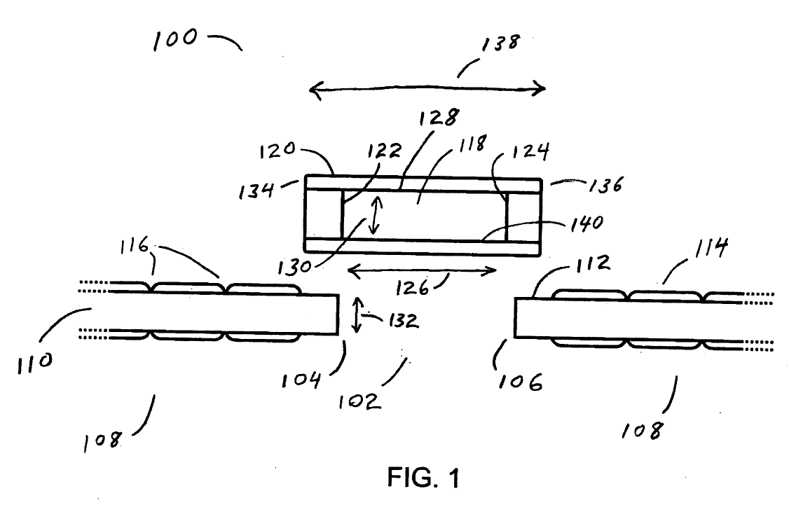

Embodiments of the present invention generally relate to devices and methods for repairing damaged nerves and for establishing interfaces among nerves, other biological cell types and electronic devices. An individual nerve cell can be referred to as a neuron, and a nerve as a functional collection of one or more neuron. FIG. 1 illustrates an ion conduit 100 in accordance with embodiments of the present invention for bridging a gap 102 between a first severed end 104 and a second severed end 106 of a damaged axon 108. The axon 108 can include axoplasm 110 within a cell membrane 112. Myelin 114 sheaths the undamaged portions of the axon 108. The myelin 114 may be discontinuous along the axon 108, interrupted periodically at Nodes of Ranvier 116.

Transmission of nerve impulses along an undamaged axon may include the transport of ions, including sodium ions, potassium ions, and other ions, through the wall of the axon. The myelin 114 can provide a barrier to the movement of ions therethrough. That is, the myelin 114 can function as an ion insulator. Ions are comparatively free to move through the axoplasm 110. That is, the axoplasm 110 can function as an ion conductor, for example, along the axon 108 between the Nodes of Ranvier 116. When an axon is severed, as illustrated in FIG. 1, the ionic conduction path, and therefore the nerve impulse, is interrupted. Other damage, such as a crushing injury, can also interrupt the transmission of nerve impulses. In either case, ion conduits in accordance with embodiments of the present invention can be used to bridge a damaged portion of an axon.

The ion conduit 100 may be adapted to transport cations including, but not limited to, potassium and sodium, between the damaged ends 104 and 106, to restore nerve function. In one embodiment, the ion conduit may be adapted to transport calcium ions. The ion conduit 100 can include a central elongated ion conductor 118 for conducting ions. The ion conductor 118 may be at least partially surrounded (i.e., jacketed) by an ion insulator 120 (i.e., ion insulating jacket). The ion conductor 118 can include a first end 122, a second end 124, and a conductor length 126 therebetween. The ion conductor 118 can also include an outer surface 128 and a conductor cross section 130. In one embodiment, the conductor cross section 130 may be adapted to resemble a cross section 132 of the severed axon 108. In one embodiment, the conductor cross section 130 may be substantially circular, and the outer surface 128 may be substantially cylindrical. In one embodiment, a diameter of a cylindrical ion conductor substantially equals a diameter of the axon 108. In another embodiment, the diameter of a cylindrical ion conductor can be larger than the diameter of the axon 108. In yet another embodiment, the ion conductor 118 can include a noncircular cross section 130. In still another embodiment, the ion conductor may be branched.

The ion insulator 120 can include a first insulator end 134, a second insulator end 136, an insulator length 138, and an inner surface 140. In one embodiment, the inner surface 140 of the ion insulator 120 can be adapted to conform to the outer surface 128 of the ion conductor 118. In another embodiment, the inner surface 140 of the ion insulator 120 may be adhered to the outer surface 128 of the ion conductor 118. In yet another embodiment, a unitary construction can include the ion conductor 118 and the ion insulator 120, where the unitary construction has an ionically insulating portion substantially surrounding an ion conducting portion, for example.

The ion conduit 100 may be adapted for coupling to the damaged axon 108 to functionally restore ion conduction between the first 104 and the second end 106. Means for reassociating severed ends of an axon are known in this art. For example, hollow coupling sleeves have been used to receive damaged nerve ends, thereby providing physical support and a conductor along which an axon might heal to restore functioning of the neuron. Known methods for joining nerve tissue also include thermal and chemical tissue welding or adhesion, and the application of pins and braces to retain damaged nerve endings in desired relative positions to encourage healing. Any suitable type of coupling mechanism or technique can be employed.

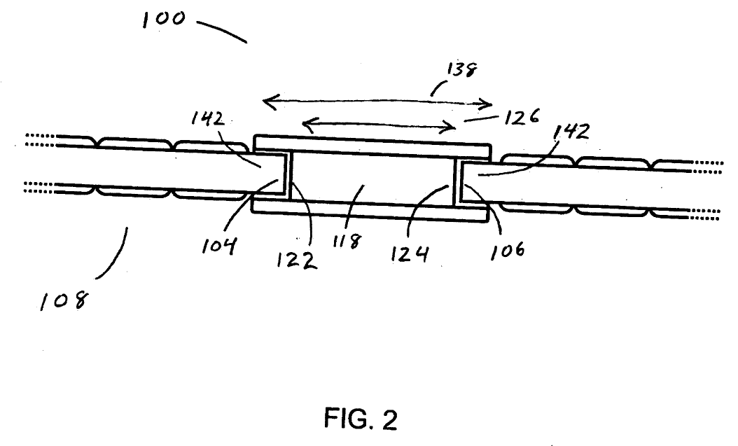

The ion conduit 100 can be coupled to the damaged ends 104 and 106 of the axon 108 by any means that can provide ion conduction at the interface between the ion conductor 118 and the axon 118 without causing substantial further damage to the axon 108 or adjacent tissue. FIG. 2 illustrates the first end 122 and the second end 124 of the ion conduit 100 coupled to the respective first severed end 104 and second severed end 106 of the axon 108. That is, the ion conduit can be implanted as a neural bridge. In the particular example shown in FIG. 2, the length 138 of the ion insulator 120 may be greater than length 126 of the ion conductor 118, forming one or more receptacles 142 in the ion conduit 100 for receiving a respective damaged end 104 or 106. In one embodiment, at least one of severed ends 104 and 106 can be inserted into a respective receptacle 142 using standard surgical techniques. In one embodiment, the ion insulator 120 can be elastometic and may be stretched diametrically to receive a respective severed end 104 or 106 as a step in a surgical procedure to repair the axon 108. Other types of insulators can be used. In another embodiment, a biological adhesive can be used to couple the ion conduit 100 to one of the first 104 and the second damaged end 106. In yet another embodiment, the ion conduit 100 can be secured to one of the first 104 or the second damaged end 106 of the axon 108 using at least one of a pin and a brace.

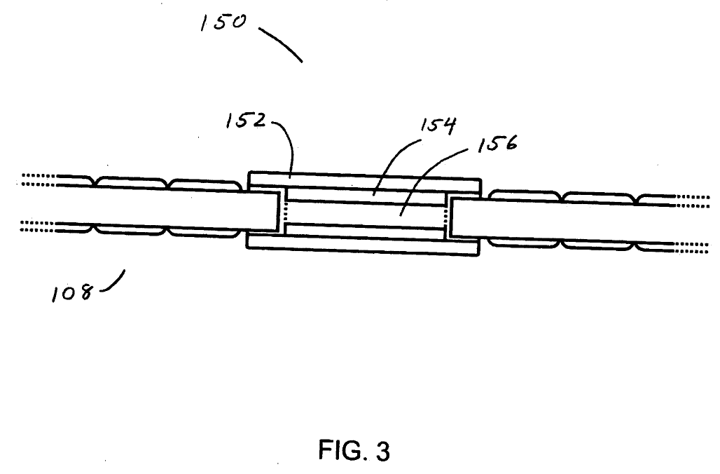

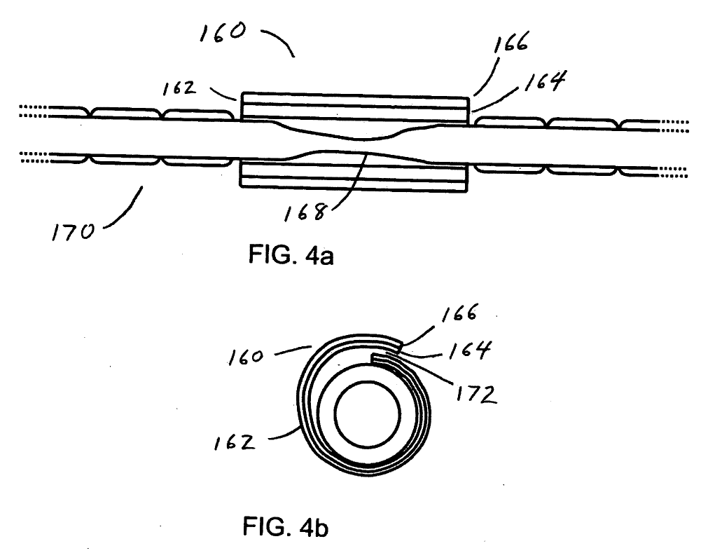

In accordance with embodiments of the present invention, an ion conduit including ion conducting and ion insulating portions can have any physical form that supports the restoration of functionality to a damaged axon. FIG. 3 illustrates an exemplary cannulated ion conduit 150 where an ion insulator 152 can surround a cannulated ion conductor 154 including a longitudinal passage 156 through which the severed axon 108 can regrow. In yet another example, illustrated in FIGS. 4a and 4b, a neural bandage 160 including a sheet of material 162 with an ion conducting layer 164 and an ion insulating layer 166 may be wrapped about a damaged portion 168 of an axon 170 to conduct ions across the damaged portion 168. In a further embodiment, the neural bandage 160 may be self-adhesive 172, configured for sealing the neural bandage 160 longitudinally along the damaged portion 168 of the axon 170. In any of the particular examples illustrated in FIGS. 3, 4a and 4b, the ionic conduit may be substantially annular about the path of the healing neuron.

In various embodiments, the ion conductor 118, 154, 164 and the ion insulator 120, 152, 166 may be fabricated from biocompatible polymers or bio-derived materials that can be bioabsorbable or nonabsorbable. These materials can include, but are not limited to, synthetic polymers, copolymers and biomaterials known in the art for fabricating tubular sheaths for joining severed nerves. Among these materials are polyurethanes, polyesters, polyolefins, halogenated polyolefins, silicones, esters of hyalurionic acid, polymers of glycerol and a diacid, agarose, chitosan, and intestinal submucosal tissue. In one embodiment, the ion insulator 120, 152, 166 may be adapted to transport cellular nutrients between surrounding extracellular fluid and the axon 108, 170. In another embodiment, at least one of the ion conductor 118, 154, 164 and the ion insulator 120, 152, 166 can be adapted to transport cellular nutrients across a damaged or severed portion of the axon 108, 170.

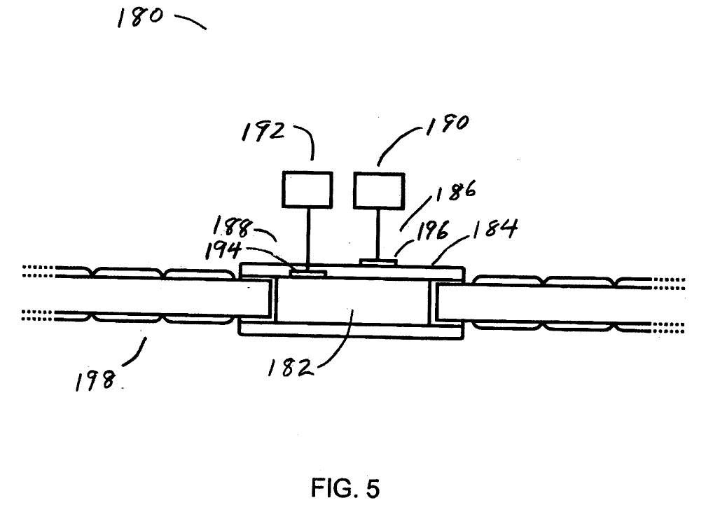

FIG. 5 illustrates exemplary embodiments of an externally ported ion conduit 180 including an ion conductor 182 and a ported ion insulator 184 having one or more interface port 186, 188. The ported ion insulator 180 can be adapted to be coupled chemically or electronically to external sensing or control devices 190, 192. In one embodiment, the one or more interface ports 186 and 188 can include one or more electrode. In one embodiment, a direct contact electrode 194 may be mounted in direct contact with the ion conductor 182. In another embodiment, a capacitively coupled electrode 196 may be spaced from the ion conductor 182 by at least a portion of the ion insulator 184, and capacitively coupled to the ion conductor 182. In one embodiment, one or more electrodes 194 and 196 may be adapted to supply an electrical potential via the sensing or control devices 190 and 192 to the ion conductor 182, thereby producing a synthetic nerve impulse, or blocking a nerve impulse in an axon 198. In embodiments including a plurality of electrodes, electrical pulses can be timed to selectively control the direction of nerve impulses passing through the ion conduit 182. In one embodiment, a plurality of electrodes may be distributed longitudinally along an ion conduit, and a combination of electrical sensing and controls contribute to propagating a nerve impulse along the conduit. In addition, the application of electric fields to damaged nerves may be used as a means to enhance or direct a direction of healing and/or regrowth. Further, an electric potential may be applied between electrodes associated with ion conduits in accordance with embodiments of the present invention to enhance healing of a damaged neuron.

In another embodiment, the one or more of interface ports 186 and 188 may be adapted for measuring electrical activity of the axon 198 (e.g., for detecting nerve impulses). In one embodiment, the one or more interface ports 186 and 188 can include an ion-selective electrode. In an alternative embodiment, the one or more interface ports 186 and 188 can include a reservoir or a sink for ions, providing an ionic concentration gradient for injecting or removing ions from the ion conductor 182. In one embodiment, the one or more interface ports 186 and 188 can include one or more sources of at least one of potassium and sodium ions. In one embodiment, the ionic concentration gradient may be induced by an externally applied electric potential. In another embodiment, measurements of electrical activity or ion concentrations at the ion conduit can be used to provide feedback to control the transmission of nerve impulses through the ion conduit 180.

An ionic conductor in accordance with embodiments of the present invention can include an ionically conductive polymer adapted to transport cations between damaged ends of an axon, or between the axon and external sensing or control devices. The transported cations can include potassium ions, sodium ions, and other ions, for example. In one embodiment, the cations include at least one of monovalent and divalent cations. In one embodiment, the ionically conductive polymer may be a block copolymer with at least one insulating block and at least one ionically conductive block, with anions immobilized in the polymer. In one embodiment, the anions may be immobilized by covalent bonding to the polymer. In one embodiment, the ionically conductive block can include one or more electronegative oxygen-containing species that is an alkyl ether or other anionic species. In one embodiment, the conductive block can include methoxy polyethylene glycol methacrylate. The ionically nonconductive block can include one or more acrylate, siloxane, polyolefin, or another subunit. In one embodiment, the ionically nonconductive block is selected from among materials described hereinabove to fabricate an ion insulator in accordance with embodiments of the present invention.

The maximum effective distance along which an ion conduit will functionally bridge a severed neuron can be dependent upon several factors, such as the ionic conductivity of the ion conductor, the diameter of the ion conductor, the health of the neuron beyond the immediate bridged damage, dispersion of the pulse at the interface between the ionic conductor and a surrounding ionic insulator, and other factors. In addition to this passive propagation of a natural nerve impulse, the inclusion of electrodes and/or an electrical interface to regenerate and additionally propagate nerve impulses through an ion conduit may extend the maximum effective distance indefinitely. In one particular example, an ion conduit may passively propagate a natural nerve impulse a maximum effective propagation distance of at least five millimeters between severed ends of an axon. In another particular example, an ion conduit including regularly spaced electrodes along its length may propagate a nerve impulse a maximum distance of at least 100 millimeters.

Surgical methods for implanting an ion conduit in accordance with embodiments of the present invention to repair nerve damage can include standard neurosurgical methods for patient and/or surgical site preparation. Ion conduits having receptacles for receiving severed nerve ends may be implanted using standard surgical methods for implanting known nerve sleeves or nerve guides. Further, chemical biological treatments of the surgical site, including, for example, the introduction of medicants to enhance healing, may also follow known surgical methods. Ion conduits in accordance with embodiments of the present invention may also be preloaded with one or more medicant for continued release post-surgery. Electrical stimulation of the ion conduit, for example using electrodes associated with the ion conduit, may also be used to control dispensing of the one or more medicant from the ion conduit.

Embodiments of the present invention have many advantages, including, but not limited to advantages associated with improved short term reestablishment of neuronal function, as well as long term healing of nerves following surgery to repair nerve damage. Ion conduits in accordance with embodiments of the present invention can emulate the ion conduction of a natural nerve impulse along an axon. Implantation of ion conduits in accordance with embodiments of the present invention may restore nerve function across a damaged portion of an axon relatively quickly, or immediately post surgery. In addition, ion conduits in accordance with embodiments of the present invention that include a passage for nerve regrowth may provide an optimal environment for healing, while also providing temporary functionality of the nerve via nerve impulse conduction along the annular ion conductor about the path of the healing.

Another advantage of ion conduits in accordance with embodiments of the present invention is that such ion conduits may be used to combine known methods for enhancing a neuron's opportunity to heal. For example, an ion conduit in accordance with embodiments of the present invention can concurrently provide any combination of: a conduit for guiding the healing of a damaged neuron, a controllable source of healing medicants, electrical stimulation to induce healing, and ongoing functionality of the healing neuron through ion conduction in a conductive polymer. Yet another advantage of embodiments of the present invention is the provision of a prosthetic bridge between severed ends of an axon that may temporarily restore neuronal function while other healing takes place, while decisions regarding additional therapies are being made, or that may provide a permanent replacement of an irreparably damaged axonal segment.

Although the invention has been described with respect to specific embodiments thereof, these embodiments are merely illustrative, and not restrictive of the invention.

In the description herein, numerous specific details are provided, such as examples of components and/or methods, to provide a thorough understanding of embodiments of the present invention. One skilled in the relevant art will recognize, however, that an embodiment of the invention can be practiced without one or more of the specific details, or with other apparatus, systems, assemblies, methods, components, materials, parts, and/or the like. In other instances, well-known structures, materials, or operations are not specifically shown or described in detail to avoid obscuring aspects of embodiments of the present invention.

Reference throughout this specification to “one embodiment”, “an embodiment”, or “a specific embodiment” means that a particular feature, structure, or characteristic described in connection with the embodiment is included in at least one embodiment of the present invention and not necessarily in all embodiments. Thus, respective appearances of the phrases “in one embodiment”, “in an embodiment”, or “in a specific embodiment” in various places throughout this specification are not necessarily referring to the same embodiment. Furthermore, the particular features, structures, or characteristics of any specific embodiment of the present invention may be combined in any suitable manner with one or more other embodiments. It is to be understood that other variations and modifications of the embodiments of the present invention described and illustrated herein are possible in light of the teachings herein and are to be considered as part of the spirit and scope of the present invention.

Embodiments of the invention may be implemented by using a programmed general purpose digital computer, by using application specific integrated circuits, programmable logic devices, field programmable gate arrays, optical, chemical, biological, quantum or nanoengineered systems, components and mechanisms may be used. In general, the functions of embodiments of the present invention can be achieved by any means as is known in the art. Further, distributed, or networked systems, components, and/or circuits can be used. Communication, or transfer, of data may be wired, wireless, or by any other means.

It will also be appreciated that one or more of the elements depicted in the drawings/figures can also be implemented in a more separated or integrated manner, or even removed or rendered as inoperable in certain cases, as is useful in accordance with a particular application. It is also within the spirit and scope of the present invention to implement a program or code that can be stored in a machine-readable medium to permit a computer to perform any of the methods described above.

Additionally, any signal arrows in the drawings/FIGs should be considered only as exemplary, and not limiting, unless otherwise specifically noted. Furthermore, the term “or” as used herein is generally intended to mean “and/or” unless otherwise indicated. Combinations of components or steps will also be considered as being noted, where terminology is foreseen as rendering the ability to separate or combine is unclear.

As used in the description herein and throughout the claims that follow, “a”, “an”, and “the” includes plural references unless the context clearly dictates otherwise. Also, as used in the description herein and throughout the claims that follow, the meaning of “in” includes “in” and “on” unless the context clearly dictates otherwise.

The foregoing description of illustrated embodiments of the present invention, including what is described in the Abstract, is not intended to be exhaustive or to limit the invention to the precise forms disclosed herein. While specific embodiments of, and examples for, the invention are described herein for illustrative purposes only, various equivalent modifications are possible within the spirit and scope of the present invention, as those skilled in the relevant art will recognize and appreciate. As indicated, these modifications may be made to the present invention in light of the foregoing description of illustrated embodiments of the present invention and are to be included within the spirit and scope of the present invention.

Thus, while the present invention has been described herein with reference to particular embodiments thereof, a latitude of modification, various changes and substitutions are intended in the foregoing disclosures, and it will be appreciated that in some instances some features of embodiments of the invention will be employed without a corresponding use of other features without departing from the scope and spirit of the invention as set forth. Therefore, many modifications may be made to adapt a particular situation or material to the essential scope and spirit of the present invention. It is intended that the invention not be limited to the particular terms used in following claims and/or to the particular embodiment disclosed as the best mode contemplated for carrying out this invention, but that the invention will include any and all embodiments and equivalents falling within the scope of the appended claims.

What is claimed is:

1. An ion conduit, comprising:
an elongated central member having a first end, a second end, a central axis therebetween, and a substantially cylindrical outer surface about the central axis, the elongated central member being adapted to conduct ions along the central axis between the first and second ends; and
an ionically insulating jacket surrounding at least a portion of the substantially cylindrical outer surface,
wherein at least one of the first and second ends is adapted to be ionically coupled to a neuron of a living animal.

2. The ion conduit of claim 1, wherein the ions include sodium ions.

3. The ion conduit of claim 1, wherein the ions include potassium ions.

4. The ion conduit of claim 1, wherein the at least one of the first and second ends is configured to be ionically coupled to a severed end of the neuron.

5. The ion conduit of claim 1, wherein the elongated central member includes an ionically conductive polymer.

6. The ion conduit of claim 5, wherein the ionically conductive polymer includes a block copolymer.

7. The ion conduit of claim 5, wherein the ionically conductive polymer includes methoxy polyethylene glycol methacrylate.

8. The ion conduit of claim 1, wherein each of the elongated central member and the ionically insulating jacket includes a biocompatible polymer.

9. The ion conduit of claim 1, wherein at least one of the elongated central member and the ionically insulating jacket is configured to be bioabsorbable.

10. The ion conduit of claim 1, further comprising one or more interruptions in the ionically insulating jacket between a distal and a proximal end of the first and second ends, the one or more interruptions being adapted to couple ions through the substantially cylindrical outer surface.

11. The ion conduit of claim 1, further comprising a recess in at least one of the first and second ends configured to receive a portion of the neuron.

12. The ion conduit of claim 1, further comprising a longitudinal passage configured for nerve growth, the longitudinal passage extending between the first and second ends.

13. The ion conduit of claim 1, further comprising one or more electrodes electrically coupled to at least one of the elongated central member and the ionically insulating jacket.

14. The ion conduit of claim 1, wherein the other of the at least one of the first and second ends is adapted to be coupled to an electrical interface configured to convert between an ionic current and an electrical current.

15. The ion conduit of claim 1, further comprising a sensor configured to sense an action potential within the elongated central member.

16. The ion conduit of claim 1, wherein at least one of the elongated central member and the ionically insulating jacket is adapted to transport a cellular nutrient between a nutrient source and the neuron.

17. An implant configured to bridge a gap in a severed nerve of a living being, the implant comprising at least one ionically conductive elongated strand having a first end, a second end, an ionically conductive polymeric core, an ionically insulating jacket about at least a portion of the ionically conductive polymeric core, at least one of the first and second ends being adapted to ionically couple to the severed nerve.

18. The implant of claim 17, wherein the at least one ionically conductive elongated strand includes at least two ionically conductive elongated strands.

19. The implant of claim 18, wherein one of the at least two ionically conductive elongated strands is configured to conduct ions in a first direction between the first and second ends and another of the at least two ionically conductive elongated strands is configured to conduct ions in an opposite direction between the first and second ends.

20. The implant of claim 17, further comprising a plurality of electrodes configured to modify an action potential in the at least one ionically conductive elongated strand.

21. A neural bandage, comprising a multilayer sheet including an ionically conductive layer and an ionically insulating layer, each of the ionically conductive layer and the ionically insulating layers being substantially biocompatible, the multilayer sheet being flexible enough to wrap about a damaged portion of a nerve to form a passage about the nerve.

22. The neural bandage of claim 21, further comprising an adhesive configured to maintain positions of overlapping portions of the neural bandage with respect to one another about the nerve.

23. A method for repairing damage to a neuron in a living animal, the method comprising:
providing an elongated ion conduit having a first end, a second end, and a length therebetween, and an ionically conducting portion and an ionically insulating portion, each extending along the length substantially from the first end to the second end; and
positioning the elongated ion conduit to ionically connect undamaged portions of the neuron, thereby bridging the damage.

24. The method of claim 23, wherein the positioning the elongated ion conduit comprises connecting at least one of the first and second ends to a severed end of the neuron.

25. The method of claim 23, wherein the positioning the elongated ion conduit comprises establishing a passage through which a growing neuron reconnects a first severed end of the neuron to a second severed end of the neuron.
